(19) United States
(12) Patent Application Publication
(10) Pub. No.: US 2008/0260816 A1
(43) Pub. Date: Oct. 23, 2008

(54) Particulate Lipid Pharmaceutical Composition

(76) Inventors: Bengt Herslof, Stockholm (SE); Per Tingvall, Norberg (SE); Anna De Kornfeldt, Vasteras (SE)

Correspondence Address: NIXON & VANDERHYE, PC, 901 NORTH GLEBE ROAD, 11TH FLOOR, ARLINGTON, VA, 22203, US

(21) Appl. No.: 12/066,154

(22) PCT Filed: Sep. 11, 2006
(86) PCT No.: PCT/SE06/01033

§ 371 Date: May 7, 2008

(30) Foreign Application Priority Data
Sep. 16, 2005 (SE) 0502043-3

(51) Int. Cl. A61K 9/66; A61K 9/16; A61K 9/26

(52) U.S. Cl. 424/455; 424/490; 424/497; 424/498; 424/493; 424/494; 424/474

(57) ABSTRACT

A particulate lipid pharmaceutical composition comprises a particulate solid non-lipid carrier and an oil-in-water emulsion on the carrier. The emulsion comprises a dissolved or dispersed pharmacologically active agent. The oil-in-water emulsion is released from the carrier on contact with an aqueous media to form an oil-in-water emulsion in the media. Also disclosed is a method of producing the composition and a tablet containing it; sachets and capsules filled with the composition; use of the composition and the tablet as a medicine; a method of administering the composition to a patient.

### FIELD OF THE INVENTION

The present invention relates to a particulate lipid pharmaceutical composition. More specifically, the invention relates to a particulate lipid pharmaceutical composition comprising a non-lipid carrier, its use, and a method for its manufacture.

### BACKGROUND OF THE INVENTION

Oil-in-water emulsions for human consumption are widely used in the foodstuff industry. Due to their heterogeneous nature all emulsions are basically unstable. A frequent problem with such emulsions is physical storage stability, another microbial degradation. Therefore the respective oil-in-water emulsion is usually prepared a short time before it is used rather than stored for an extended period of time. These drawbacks in particular hinder their use in the pharmaceutical field, where requirements in regard of purity, acceptable degradation on storage, and user convenience are substantially stiffer than in the foodstuff field.

### OBJECTS OF THE INVENTION

The present invention seeks to overcome one or several of the aforementioned problems by providing a means for preparing an oil-in-water emulsion, which means is stable for long-term storage and can be easily handled in standard and non-standard industrial processes used in the pharmaceutical industry.

Further objects of the invention will be apparent from the following summary of the invention, the description of preferred embodiments thereof, and the appended claims.

### SUMMARY OF THE INVENTION

According to the present invention is provided a particulate lipid pharmaceutical composition comprising a particulate solid non-lipid carrier, an oil-in-water emulsion on the carrier and comprising a pharmacologically active agent dissolved and/or dispersed therein, the emulsion being capable of release from the carrier on contact with an aqueous media to form an oil-in-water emulsion in said media.

The pharmacologically active agent of the invention may be any agent suitable for administration in form of an oil-in-water emulsion.

According to one preferred aspect of the invention the particle size of the composition of the invention is determined by the particle size of the carrier, the composition substantially consisting of particles, each comprising a single carrier particle only to which oil-in-water emulsion adheres.

According to another preferred aspect of the invention the particle size of the composition of the invention is determined by the capability of two or more particles, each comprising a single carrier particle to which oil-in-water emulsion adheres, to form larger aggregates.

It is preferred for the particulate lipid composition of the invention to be free-flowing to enable it to be processed in equipment used in the pharmaceutical industry.

According to a basic aspect of the invention the mean weight of the particles of the composition of the invention is preferably 10 mg or lower, more preferred 1 mg or lower, most preferred 0.1 mg or lower.

According to an alternative basic aspect of the invention carrier particles of larger size are used to bring the mean weight of the particles of the composition of the invention to more than 5 mg or 10 mg or even 50 mg.

An important aspect of the invention is that the carrier must not dissolve in the oil-in-water emulsion or otherwise be substantially affected by it, this being a condition for the oil-in-water emulsion to be preserved substantially unchanged for storage and for being released from the carrier in contact with an aqueous media.

The oil-in-water emulsion of the invention comprises a non-polar lipid and a lipidic emulsifier. Suitable oil-in-water emulsions including non-polar lipids and lipidic emulsifiers for incorporation into the composition of the invention are disclosed in U.S. Pat. No. 6,517,883 (Herslöf et al.), U.S. Pat. No. 6,355,693 (Herslöf et al.), and U.S. Pat. No. 5,688,528 (Carlsson et al.), which are hereby incorporated for reference. According to an advantageous aspect of the invention the oil-in-water emulsion may comprise pharmaceutically acceptable excipients, such as antioxidant; colourant; flavouring.

The non-polar lipid of the invention is preferably triglyceride, which is solid, semi-solid, or liquid at room temperature, selected from natural, semi-synthetic and synthetic oil. Natural oils are preferably based on the combination of mainly, that is, to more than 90% by weight, preferably to more than 95% by weight, palmitic, oleic, linoleic, linolenic, and stearic esters of glycerol are preferred. Most preferred is palm oil and its equivalent confectionery fats, such as coconut oil, palm kernel oil, cocoa butter; partially hydrogenated soybean oil; partly hydrogenated rapeseed oil; sunflower oil and its equivalent liquid vegetable oils, such as soybean oil, rapeseed oil, safflower oil, olive oil, corn oil, groundnut oil, linseed oil, rice bran oil, and sesame oil; animal fats and oils, such as fish oil, butter fat, lard, tallow, their fractions and mixtures thereof. The weight ratio of non-polar lipid to emulsifier is preferably from 6:1 to 60:1, more preferred from 10:1 to 30:1.

The lipidic emulsifier of the invention can be of natural or synthetic, including semi-synthetic, origin. Particularly preferred are emulsifiers selected from mono- and diglycerides, in particular of lauric, myristic, palmitic, stearic, oleic, linoleic, and linolenic acid, their mixtures and acid esters, in particular their acetates; sorbitan esters and polysorbates; polyglycerol esters; sucrose esters; propylene glycol mono fatty acid esters; esters of lactic acid, succinic acid, fruit acid; lecithin; specific membrane lipids, such as phospholipid, galactolipid, and sphingolipid. The emulsifier of the invention is preferably selected from phospholipid-containing material, such as soy lecithin, and galactolipid-containing material, such as fractionated oat oil, of which galactolipid-containing material is most preferred. A preferred galactolipid-containing material comprises 20% by weight to 30% by weight of galactolipid, mainly digalactodiacylglycerol, and from 10% by weight to 15% by weight of other polar lipid.

The carrier of the invention is preferably of vegetable or inorganic origin. Preferably the carrier is capable of passing at least the upper part of the gastro-intestinal tract substantially unchanged. According to one preferred aspect, the carrier of the invention is substantially insoluble in water but does swell in contact with it. According to an alternative preferred aspect, the carrier of the invention is partially or fully soluble in water. Preferred carriers are comprised by the group consisting of starch, modified starch such as pre-gelatinized starch, microcrystalline cellulose, powdered cellulose, cellulose derivatives such as hydroxymethyl propyl cellulose and methyl cellulose, mannitol, sorbitol, anhydrous lactose, active carbon, other material of vegetable origin such as material originating from oat bran, rice hulls, ground seeds, etc., gums such as gum arabic, pectins, xanthans, and carrageenans. In addition to organic carrier materials inorganic carrier materials used in the pharmaceutical industry, such as sodium chloride, calcium carbonate, calcium phosphate, calcium sulphate dihydrate, amorphous silica, may be used in certain applications. It is furthermore possible to use particles of or comprising synthetic polymers as a carrier, such as poly(γ-hydroxybutyrate), polylactide, polyglycolide, poly(lactide, glycolide), and methacrylates. Polymer non-woven materials like one disclosed in U.S. Pat. No. 6,268,434 can also be used as a carrier. It is also within the scope of the invention to use mixtures of the carrier materials of the invention. In principle, any pharmaceutically acceptable solid particulate carrier material that does not interact, at least not to a substantial degree, with the oil-in-water emulsion in an irreversible manner preventing it from being released on contact with aqueous media to form an oil-in-water emulsion in said aqueous media may be used.

It is preferred for the composition of the invention to comprise from 0.1% by weight to 90% by weight of oil-in-water emulsion and from 10% to 99.9% by weight of carrier; more preferred from 0.5% by weight to 60% by weight of oil-in-water emulsion and from 99.5% by weight to 40% by weight of carrier; even more preferred from 0.5 by weight to 40% by weight, most preferred to 30% by weight of oil-in-water emulsion and from 60% by weight, most preferred from 70% by weight, to 99.5 by weight of carrier.

The term aqueous media as used herein comprises water and aqueous solutions of salts such as sodium chloride and/or of organic compounds such as glucose but also gastric fluids. It is preferred for the composition to release more than 50% by weight, more preferred more than 75% by weight, of its oil-in-water emulsion on contact with an aqueous media at a temperature of below 75° C., more preferred of below 50° C., even more preferred of below 40° C., most preferred at about 35° C.

According to an additional preferred aspect of the invention the mean particle size (number average) of the emulsion formed by contact of the composition of the invention with an aqueous media exceeds that of the emulsion used for preparing the composition of the invention on contact with the same media by less than 30%, preferably by less than 15%, most preferred by less than 10%.

According to the present invention is also disclosed a method of producing a particulate lipid pharmaceutical composition that comprises a particulate solid non-lipid carrier and an oil-in-water emulsion, the emulsion comprising a pharmacologically active agent dissolved and/or dispersed therein, the emulsion being disposed on the carrier and capable of being released from the carrier on contact with an aqueous media to form an oil-in-water emulsion in said media, comprising the steps of: (a) providing an oil-in-water emulsion in liquid form comprising a pharmacologically active agent dissolved and/or dispersed therein; (a1) alternatively providing oil-in-water emulsion in liquid form and a pharmacologically active agent; (a2) dissolving and/or dispersing the agent in the emulsion of (a1); (b) providing a particulate solid non-lipid carrier; (c) adding the oil-in-water emulsion of (a) or (a2) to the carrier over a period of time while agitating the carrier to obtain said particulate lipid composition. It is preferred for oil-in-water emulsion to be provided at a temperature of from 30° C. to 75° C. It is also preferred to cool the carrier and the product formed from the carrier during addition of the emulsion so as to keep their temperature below 30° C. The method of the invention may comprise the additional step of: (d) separating a fraction of defined particle size from said particulate lipid composition by, for instance, sieving.

The composition of the invention can be used as such as a medicine, for instance filled into a sachet containing a weighed dose of it. For administration the patient will open the sachet, pour the contents into a suitable volume of water in a beaker or drinking glass, wait for the emulsion to form, and swallow it. Alternatively a weighed amount of the composition of the invention is filled into a gelatin or other capsule that can be swallowed.

According to a further preferred aspect of the invention a weighed amount of the composition of the invention is mixed with pharmaceutical excipient, which mixture is fed into a tablet press to produce pharmaceutical tablets. The pharmaceutical excipient preferably comprises tabletting aids that easily disintegrate in aqueous solutions including gastric fluids. For this purpose the tablets may comprise a disintegrant such as sodium starch glycolate, hydroxypropyl methyl cellulose, microcrystalline cellulose, and crosslinked polyvinyl pyrrolidone. The tablets can be coated in a conventional manner to make them easy to swallow, such as by sugar coating. Because of their sensitivity to aqueous media, precautions such as by the provision of a sealing, such as a conventional shellac, HPMC, and polyvinyl acetate phthalate (PVAP) sealing, on the tablet prior to applying the sugar coat. To retain, as much as possible, the physical structure of the composition of the invention in admixture with pharmaceutical excipient on compression, preferably direct compression, into tablets, low compression forces should preferably be used to obtain tablets with a crushing strength of from about 2 kp to about 10 kp, more preferred from about 2 kp to 6 kp.

According to a further preferred aspect the composition of the invention, either in form of free flowing particles or free flowing aggregates of such particles, a gelatin or other capsule filled with the particles or aggregates, or a tablet formed from the particles or aggregates is enterically coated. The free flowing particles or aggregates are preferably coated in a fluid bed reactor. A suitable enteric coating such as cellulose acetate phthalate, polyvinyl acetate phthalate, triethanolamine cellulose acetate phthalate, hydroxypropyl methyl cellulose, hydroxypropyl methyl cellulose phthalate, hydroxypropyl methyl cellulose acetate succinate, copolymers of methyl methacrylate and ethyl acrylate with methacrylic acid, will delay the contact between the composition of the invention and gastric fluid and/or to protect the gastric mucosa from irritating components of the composition.

According to the present invention is also disclosed a method of administering a pharmacologically active agent to a patient, comprising: (o) contacting the particulate composition of the invention or a tablet formed from it with water or an aqueous media; (p) allowing an oil-in-water emulsion to form; (q) making the patient swallow the emulsion formed in step (p). The method of administration may comprise the additional step I of separating the carrier from the oil-in-water emulsion by, for instance, filtration to retain the carrier on the filter; sedimentation of the carrier, provided the carrier has a specific weight exceeding that of water or the aqueous media, respectively; skimming off, provided that the carrier has a specific weight inferior to that of water or the aqueous media, respectively.

The invention will now be described in more detail in form of a number of non-limiting embodiments.

### DETAILED DESCRIPTION OF THE INVENTION

All percentages and ratios herein are by weight.

Exemplary non-lipid carrier materials. A number of exemplary non-lipid carrier materials available on the market are listed in Table 1.

TABLE 1
Non-lipid carrier materials

| | Non-lipid carrier material | Supplier |
|---|---|---|
| A | Potato starch, Art. No. 94441.1 | Carl Roth GmbH & |
| | | Co.¹ |
| B | Corn starch, Purity 826 LBI 6545 | National Starch & |
| | | Chemical² |
| C | Pregelatinized starch, Colorcon | Colorcon³ |
| G | Microcrystalline cellulose, Avicel ® | FMC Corp.⁴ |
| | PH102 | |
| | Cellulose powder, Elcema ® P 050 | Degussa AG⁵ |
| H | Lambda-Carrageenan, Viscarin GP 209F, | FMC Corp. |
| | Lot no. 3091204B | |
| I | Xanthan, Keltrol RD, Art. No. 2107 | CPKelco⁶ |
| K | Dialciumphosphate dihydrate, DiTAB ® | Rhodia Inc.⁷ |
| | Crystalline Sorbitol, Sorbogem ® 834 | SPI Polyols Inc.⁸ |
| M | Mannitol powder | Roquette⁹ |
| N | Spray-dried lactose | Foremost Farms¹⁰ |
| | Hydroxypropyl cellulose HXF Pharm | Aqualon¹¹ |

¹Veghel, Netherlands;
²Stockholm, Sweden;
³Dartfort, Kent, UK;
⁴Decatur IL, U.S.A.;
⁵Frankfurt(Main), Germany;
⁶Bridgewater NJ, U.S.A.;
⁷Cranbury NJ, U.S.A.;
⁸New Castle DE, U.S.A.;
⁹Roquette GmbH, Frankfurt, Germany;
¹⁰Rothschild WI, U.S.A.;
¹¹Div. of Hercules Inc., Wilmington DE, U.S.A.

### EXAMPLE 1

Exemplary method of preparing the composition of the invention. An oil-in-water lipid emulsion for use in the invention is prepared by mixing weighed amounts of an oil, in which a pharmaceutically active agent has been dissolved and/or suspended, such as palm oil, an emulsifier such as fractionated oat oil, and water with a powerful mechanical mixer such as a T 18 ULTRA-TURRAX® (IKA Werke GmbH & Co. KG, Staufen, Germany. Alternatively the pharmaceutically active compound can be dissolved and/or suspended in any of oil, emulsifier and water or in the oil-in-water lipid emulsion when formed in the mixing stage. A weighed amount of the emulsion is added drop-wise to a weighed amount of the carrier in a glass flask while gently shaking the flask in intervals. At the end of addition the mixture is stirred with a spatula until apparent homogeneity.

### EXAMPLE 2

Preparation of a phenytoin composition according to the invention. Phenytoin powder (5,5-diphenylhydantoin, an antiepileptic; 3.0 g) is added to a water-in-oil emulsion 100 ml of an water-in-oil emulsion prepared from 40 g of palm oil, 3 g of EB05004K galactolecithin (LTP Lipid Technology Provider AB, Karlshamn, Sweden) and 57 ml water while stirring with an T 18 ULTRA-TURRAX® apparatus. After stirring for 10 min the mixture is slowly poured on 300 g of microcrystalline cellulose (Avicel® PH102, carrier) while stirring by hand. The lumpy product is cooled to 5° C., put on a large wire cloth No. 14 sieve (mesh opening 1.4 mm) connected to a shaking machine while manually assisting sieving/disintegration of lager aggregates. The particulate product is stored in a refrigerator. A daily maintenance dose for an adult suffering from epilepsy is one typically containing about 300 mg of phenytoin. This dose can be administered to the patient by pouring 10 g of the particulate product into a container such as a cup or drinking glass, containing about 200 ml water to release the water-in-oil emulsion and the drug, and to make the patient drink the cloudy product formed. If calcium sulphate dihydrate or amorphous silica is used as a carrier instead of cellulose, their high specific weight will make them settle in the container so that the patient can easily decant the contents or empty the container nearly to the bottom while avoiding ingestion of the carrier. Administration of phenytoin according to the invention will have a beneficial effect on the gastro-intestinal tract since phenytoin, like many other drugs, is known to irritate the gastro-intestinal mucosa, in particular if administered regularly over an extended period of time.

### EXAMPLE 3

### Preparation of Compositions of the Invention Suitable for Incorporation of a Pharmacologically Active Agent

(a) A water-in-oil emulsion prepared from 40% of palm oil and 3% of EB05004K galactolecithin were added to Aerosil® 200 (batch 3722 AA-2 (Degussa) in a ratio of 3:7 while gently stirring. A powderous product was obtained.

(b) A water-in-oil emulsion prepared from 40% of palm oil and 3% of EB05004K galactolecithin was added to hydroxypropyl methyl cellulose (HPMC, PharmaCoat 615 batch 307412 (Shin-Etsu Chemical Co, Ltd., Japan) in a ratio of 1:1 while gently stirring. A powderous product was obtained.

### EXAMPLE 4

### Release of the Water-in-Oil Emulsion from the Powderous Product of Example 3

10 g of the respective product ((a) or (b)) was poured into 100 ml of water while stirring by hand.

(a) The release observed during the first ten minutes after addition to water (22° C.) was small.

In contrast, the release was good in water of a temperature of 60° C. Microscopy showed smaller and larger oil drops to be present, as well as areas of coalescence. Mild centrifugation at 629 g rendered a bottom layer of aerosil particles, an intermediate layer of small and larger oily particles, and a small whitish top layer. Micro-centrifugation rendered a bottom layer, a clear intermediate layer, and a whitish top layer.

(b) The release observed during the first ten minutes after addition to water (22° C.) was small. Large lumps of lipid material could be seen. Release at 60° C. was good but slower than with composition (a). Microscopy showed a few particles of various size. Mild centrifugation at 629 g resulted in an opaque liquid with a white top phase of numerous particles. Micro-centrifugation at 14000 g rendered two phases as at mild centrifugation; no bottom layer could be observed.

1. Particulate lipid pharmaceutical composition comprising a particulate solid non-lipid carrier, an oil-in-water emulsion on the carrier and comprising a pharmacologically active agent dissolved and/or dispersed therein, the emulsion being capable of release from the carrier on contact with an aqueous media to form an oil-in-water emulsion in said media.

2. The composition of claim 1, the particle size of which is determined by the particle size of the carrier, the composition substantially consisting of particles, each comprising a single carrier particle only to which oil-in-water emulsion adheres.

3. The composition of claim 2, in free flowing form.

4. The composition of claim 1, the particle size of which is determined by the capability of two or more particles, each comprising a single carrier particle only to which oil-in-water emulsion adheres, to form larger aggregates.

5. The composition of claim 4, in free flowing form.

6. The composition of claim 1, wherein the carrier is insoluble in the oil-in-water emulsion.

7. The composition of claim 1, wherein the oil-in-water emulsion comprises a non-polar lipid and a lipidic emulsifier.

8. The composition of claim 1, wherein the oil-in-water emulsion comprises one or more pharmaceutically acceptable excipients.

9. The composition of claim 1, wherein the non-polar lipid is a triglyceride, which is solid, semi-solid, or liquid at room temperature, selected from natural, semi-synthetic oil, synthetic oil, and mixtures thereof.

10. The composition of claim 9, wherein the natural oil comprises more than 90% by weight of palmitic, oleic, linoleic, linolenic, and/or stearic ester of glycerol.

11. The composition of claim 9, wherein the natural oil is selected from palm oil and its equivalent confectionery fats, such as coconut oil, palm kernel oil, cocoa butter; partially hydrogenated soybean oil; partly hydrogenated rapeseed oil; sunflower oil and its equivalent liquid vegetable oils, such as soybean oil, rapeseed oil, safflower oil, olive oil, corn oil, groundnut oil, linseed oil, rice bran oil, and sesame oil; animal fats and oils, such as fish oil, butter fat, lard, tallow, their fractions and mixtures thereof.

12. The composition of claim 1, wherein the weight ratio of non-polar lipid to emulsifier is preferably from 6:1 to 60:1.

13. The composition of claim 12, wherein the weight ratio of non-polar lipid to emulsifier is from 10:1 to 30:1.

14. The composition of claim 1, wherein the emulsifier is selected from natural and synthetic, including semi-synthetic, emulsifier, and their mixtures.

15. The composition of claim 14, wherein the emulsifier is selected from mono- and diglyceride, in particular of lauric, myristic, palmitic, stearic, oleic, linoleic, and linolenic acid, their mixtures and esters, in particular their acetates;
sorbitan esters and polysorbates; polyglycerol esters; sucrose esters; propylene glycol mono fatty acid esters; esters of lactic acid, succinic acid, fruit acid; lecithins; specific membrane lipids, such as phospholipids, galactolipids, and sphingolipids; and mixtures thereof.

16. The composition of claim 14, wherein the emulsifier is selected from phospholipid containing material, such as soy lecithin.

17. The composition of claim 14, wherein the emulsifier is selected from galactolipid containing material, such as fractionated oat oil.

18. The composition of claim 17, wherein the galactolipid containing material comprises 20% by weight to 30% by weight of galactolipid and from 10% by weight to 15% by weight of other polar lipid.

19. The composition of claim 1, wherein the carrier is of vegetable or inorganic origin.

20. The composition of claim 19, wherein the carrier is selected from starch, modified starch such as pre-gelatinized starch, microcrystalline cellulose, powdered cellulose, cellulose derivatives such as hydroxymethylpropyl cellulose and methyl cellulose, mannitol, sorbitol, anhydrous lactose, active carbon, other material of vegetable origin such as material originating from oat bran, rice hulls, ground seeds, and similar, gums such as gum arabic, pectins, xanthans, and carrageenan.

21. The composition of claim 19, wherein the carrier is selected from sodium chloride, calcium carbonate, calcium phosphate, calcium sulphate dihydrate, amorphous silica.

22. The composition of claim 19, wherein the carrier comprises synthetic polymer.

23. The composition of claim 22, wherein the synthetic polymer comprises poly (γ-hydroxybutyrate), polylactide, polyglycolide, poly (lactide, glycolide), methacrylate.

24. The composition of claim 1, wherein the carrier is capable of passing the upper part of the gastro-intestinal tract substantially unchanged.

25. The composition of claim 1, wherein the carrier is substantially insoluble in water but may swell in contact with water.

26. The composition of claim 1, wherein the carrier is partially or fully soluble in water.

27. The composition of claim 1 comprising from 0.1% by weight to 90% by weight of oil-in-water emulsion and from 10% to 99.9% by weight of carrier.

28. The composition of claim 27, comprising from 0.5% by weight to 60% by weight of oil-in-water emulsion and from 40% by weight to 99.5% by weight of carrier.

29. The composition of claim 27, comprising from 0.5 by weight to 40% by weight of oil-in-water emulsion, and from 60% by weight to 99.5% by weight of carrier.

30. The composition of claim 27, comprising from 0.5 by weight to 30% by weight of oil-in-water emulsion, and from 70% by weight to 99.5% by weight of carrier.

31. The composition of claim 1, capable of releasing more than 50% by weight of its oil-in-water emulsion on contact with an aqueous media at a temperature of below 75° C.

32. The composition of claim 31, wherein the release temperature is below 50° C.

33. A method of producing a particulate lipid pharmaceutical composition that comprises a particulate solid non-lipid carrier and an oil-in-water emulsion, the emulsion comprising a pharmacologically active agent dissolved and/or dispersed therein, the emulsion being disposed on the carrier and capable of being released from the carrier on contact with an aqueous media to form an oil-in-water emulsion in said media, comprising the steps of:
(a) providing an oil-in-water emulsion in liquid form comprising a pharmacologically active agent dissolved and/or dispersed therein;
(a1) alternatively providing an oil-in-water emulsion in liquid form and a pharmacologically active agent;
(a2) dissolving and/or dispersing the agent in the emulsion of (a1)
(b) providing a particulate solid non-lipid carrier;
(c) adding the oil-in-water emulsion of (a) or (a2) to the carrier over a period of time while agitating the carrier to obtain said particulate lipid composition.

34. The method of claim 33, wherein the oil-in-water emulsion is added at a temperature of from 30° C. to 75° C.

35. The method of claim 34, comprising cooling the carrier and the product formed from the carrier during addition of the emulsion so as to keep their temperature below 30° C.

36. The method of claim 33, comprising the step (d) of separating a fraction of defined particle size from said particulate lipid composition.

37. The method of claim 36, wherein separation is by sieving.

38. The method of claim 33, comprising the step (e) of coating the particulate lipid composition.

39. The method of claim 38, wherein the coat provided on the composition is an enteric coat or a sugar coat.

40. Use of the composition of claim 1 as a medicine.

41. A sachet filled with the composition of claim 1.

42. A capsule filled with the composition of claim 1.

43. A method of producing a pharmaceutical tablet comprising: (i) dry mixing the composition of claim 1 and pharmaceutical excipient to produce a free flowing mixture; (ii) feeding the mixture to a tablet press;
(iii) compressing the mixture to form a tablet.

44. The method of claim 43, wherein the compression force in step (iii) is controlled so as to produce a tablet having a crushing strength of from 2 to 10 kp.

45. The method of claim 43, comprising the step (iv) of coating the tablet.

46. The method of claim 45, wherein the coat provided to the tablet is an enteric coat or a sugar coat.

47. A method of administering a pharmacologically active agent to a patient comprising (o) contacting the composition of claim 1 with water or an aqueous media; (p) allowing the composition to form an oil-in-water emulsion; (q) making the patient swallow the emulsion formed in step (p).

48. The method of claim 47, comprising the additional step (r) of separating the carrier from said oil-in-water emulsion.

49. The method of claim 48, wherein separation is by sedimentation of the carrier.

50. The method of claim 48, wherein separation is by filtration so as to retain the carrier on the filter.

51. The method of claim 48, wherein separation is by skimming the carrier off.
